(19) United States
(12) Patent Application Publication
(10) Pub. No.: US 2008/0011002 A1
(43) Pub. Date: Jan. 17, 2008

(54) Air Conditioning System For Rooms Required To Be Sterilized

(76) Inventors: Naotake Eidai, Toyama (JP); Toshihiro Nogame, Toyama (JP); Noritomo Matsuki, Toyama (JP); Miyoshi Katou, Tokyo (JP); Yoshihide Wakayama, Tokyo (JP); Shinkou Miyake, Tokyo (JP); Noriyoshi Kosaka, Kanagawa (JP); Shinichi Okuda, Kanagawa (JP); Fumitoshi Kadowaki, Kanagawa (JP)

Correspondence Address: TOWNSEND & BANTA;c/o PORTFOLIO IP, PO BOX 52050, MINNEAPOLIS, MN, 55402, US

(21) Appl. No.: 11/596,739

(22) PCT Filed: May 2, 2005
(86) PCT No.: PCT/JP05/08282

§ 371 Date: Sep. 10, 2007

(30) Foreign Application Priority Data
May 18, 2004 (JP) 2004-14654

(51) Int. Cl. F24F 3/16; A61L 2/20

(52) U.S. Cl. 62/78; 422/28

(57) ABSTRACT

In the conventional fumigation of the rooms required to be sterilized, formaldehyde is general used, but formaldehyde has such problems as carcinogenicity and residual tendency. It is also proposed to combine fumigation and air conditioning for automation, but air conditioning and fumigation cannot be performed concurrently. The present invention proposes an air conditioning system for the rooms required to be sterilized, comprising an air conditioning path consisting of an conditioned air supply path extending from the discharge side of an air conditioner to the air supply openings of the rooms required to be sterilized and an indoor air suction path extending from the air suction openings of the rooms required to be sterilized to the suction side of the air conditioner; a bypath for bypassing the air conditioner; an ozone supply path extending from an ozone generation unit to a switching mechanism and further branching into the ozone supply paths connected with said conditioned air supply path and said bypath; an ozone decomposition path with an ozone decomposition unit, in parallel with the bypath; an air discharge path with an air discharge fan, connected with said indoor air suction path or with the rooms required to be sterilized; and a switching mechanism for switching between said air conditioning path, said bypath, said ozone decomposition path and said air discharge path.

### TECHNICAL FIELD

The present invention relates to an air conditioning system for the rooms required to be sterilized, for example, the preparation rooms of an injection preparation factory or the sterile production rooms of a food factory.

### BACKGROUND ART

For the rooms required to be sterilized, for example, in the preparation rooms of an injection preparation factory or the sterile production rooms of a food factory, a sterilizing gas is periodically introduced into such rooms for fumigation to internally sterilize them. As the sterilizing gas, formaldehyde is generally used.

Automatic fumigation systems using a sterilizing gas such as formaldehyde are already practically used. For example, patent document 1 describes a system in which the rooms concerned are respectively provided with an air supply duct and an air discharge duct exclusive for air conditioning separately from a gas supply duct and a gas discharge duct exclusive for fumigation, so that fumigation with formaldehyde or the like and air conditioning can be performed automatically in the rooms. In this system, while fumigation is performed in the rooms, the dampers of air supply and discharge ducts for air conditioning are closed, and while air conditioning is performed, the dampers of gas supply and discharge ducts for fumigation are closed.

On the other hand, patent document 2 describes an apparatus for internally disinfecting and sterilizing a room using ozone gas as a sterilizing gas. Patent document 2 discloses an ozone sterilization apparatus used for internally disinfecting and sterilizing a rearing chamber, etc. of a laboratory animal facility. In this apparatus, an air inlet with a screen-like pre-filter is installed below in a housing, and an air supply fan is installed beside the air inlet in the housing. Above the discharge opening of the fan, a diffusion plate and a switching damper are installed, and above the switching damper, a honeycomb structure formed of active carbon fibers, having an ozone decomposition section and a bypath section is installed. Further on it, a medium performance air filter for removing the dust in air is installed. Moreover, at the top in the housing, an ozone generator and a humidifier are installed separately from each other, and above them, an ozone gas supply opening with a louver and a humidified air supply opening with a louver are provided. This housing with castors below is moved to a room in need of it and manually operated to blow ozone and humidified air into the room, etc. for disinfecting and sterilizing a limited region in the room.
Patent document 1: JP8-6936B
Patent document 2: JP5-146497A

### DISCLOSURE OF THE INVENTION

Problems to be Solved by the Invention

As described above, fumigation of a room with formaldehyde is generally performed, but it has the following problems.
a. Formaldehyde is carcinogenic and tends to remain.
b. Because of the above properties, the leak of formaldehyde from a fumigated room to another room is very dangerous, and the greatest care must be taken for its handling, for example, by containing the fumigation in an area and treating the exhaust air after completion of fumigation
c. Automatic fumigation systems like that of patent document 1 are practically used. However, since it is necessary to suspend air conditioning during fumigation, such systems are not suitable for rooms requiring frequent fumigation.
d. The air conditioning system as a whole including the air conditioner cannot be sterilized.

On the other hand, in the case where a device for generating ozone gas is carried into a room to be disinfected and sterilized, for manually supplying ozone gas as described in patent document 2 or in the case where a sterilizing chemical liquid such as alcohol is manually sprayed for internally disinfecting and sterilizing a room, the following problems arise.
a. If ozone gas is used for sterilization, the interior materials and air conditioner components in the room concerned are likely to deteriorate and corrode.
b. Manual spraying of a chemical liquid is troublesome and dangerous and has a problem in view of reliability of sterilization work.
c. Since only a limited region can be sterilized, cross contamination with non-sterilized portions cannot be avoided. Further, in case of ozone sterilization, the materials of the region concerned deteriorate as described above.

The object of this invention is to solve the aforesaid problems.

Means for Solving the Problems

To solve the aforesaid problems, this invention proposes an air conditioning system for the rooms required to be sterilized, comprising an air conditioning path consisting of a conditioned air supply path extending from the discharge side of an air conditioner to the air supply openings of the rooms required to be sterilized and an indoor air suction path extending from the air suction openings of the rooms required to be sterilized to the suction side of the air conditioner; a bypath for bypassing the air conditioner; an ozone supply path extending from an ozone generation unit to a switching mechanism and further branching into the paths connected with said conditioned air supply path and said bypath; an ozone decomposition path with an ozone decomposition unit, in parallel with the bypath; an air discharge path with an air discharge fan, connected with said indoor air suction path or with the rooms required to be sterilized; and a switching mechanism for switching between said air conditioning path, said bypath, said ozone decomposition path and said air discharge path.

Further in said air conditioning system, this invention proposes to install an ozone decomposition catalyst at a position upstream of the heat exchange coils of the air conditioner. Meanwhile, the ozone decomposition catalyst can be installed within the air conditioner or outside the air conditioner, namely, at a position upstream of the air conditioner, if it is installed at a position upstream of the heat exchange coils.

Furthermore in said air conditioning system, this invention proposes to use materials with an ozone exposure resistance ratio of 0.75 or more in exposure to an ozone concentration of 200 ppm for 500 hours as the component materials destined to contact ozone gas, of the system.

Meanwhile, the ozone exposure resistance ratio refers to the ratio of the physical values (strength, hardness, ductility, color, rust generation, etc.) of a material exposed to zone to the physical values of the material not exposed to ozone. That is,
Ozone exposure resistance ratio=Physical values after exposure/Physical values before exposure

The physical values of a material include tensile strength, tensile ductility, surface hardness, chrominance change, etc. if the material is organic, or include tensile strength, rust generation, chrominance change, etc. if the material is inorganic. These values are measured according to JIS.

Furthermore in said air conditioning system, this invention proposes to use resin materials with a resin saturation degree of 70% or more as the resin materials destined to contact ozone gas, of the system.

Furthermore in said air conditioning system, this invention proposes to use inorganic materials, in the surfaces of which one or more of Al, Cr, Zn, Ni and Si account for 3% or more of the material concerned, as the inorganic materials of the system, which are destined to contact ozone.

Effects of the Invention

In the air conditioning system of this invention, if the switching mechanism selects the air conditioning path through the air conditioner, for performing air conditioning operation, ozone gas can be supplied from the ozone generation unit through the switching mechanism to the conditioned air supply path, for sterilizing the rooms concerned by fumigation with ozone and also for sterilizing the air conditioning system including the air conditioner and the piping system as a whole.

In this case, if the ozone decomposition catalyst is installed as required at a position upstream of the heat exchange coils the corrosion of the heat exchange coils by ozone can be prevented.

Further in this invention, if the bypath for bypassing the air conditioner is selected instead of the air conditioning path through the air conditioner, ozone gas can be supplied to the bypath from the ozone generation unit through the switching mechanism, for sterilizing the rooms concerned and the piping system while air conditioning is kept suspended.

In this invention, if adequate materials are selected as the component materials destined to contact ozone gas, of the system, the deterioration of materials can be prevented, and contaminants such as dust generated by the deterioration of materials can be reduced.

### EXAMPLE 1

An example of the air conditioning system allowing sterilization by ozone gas of this invention will be described below in reference to the attached drawings.

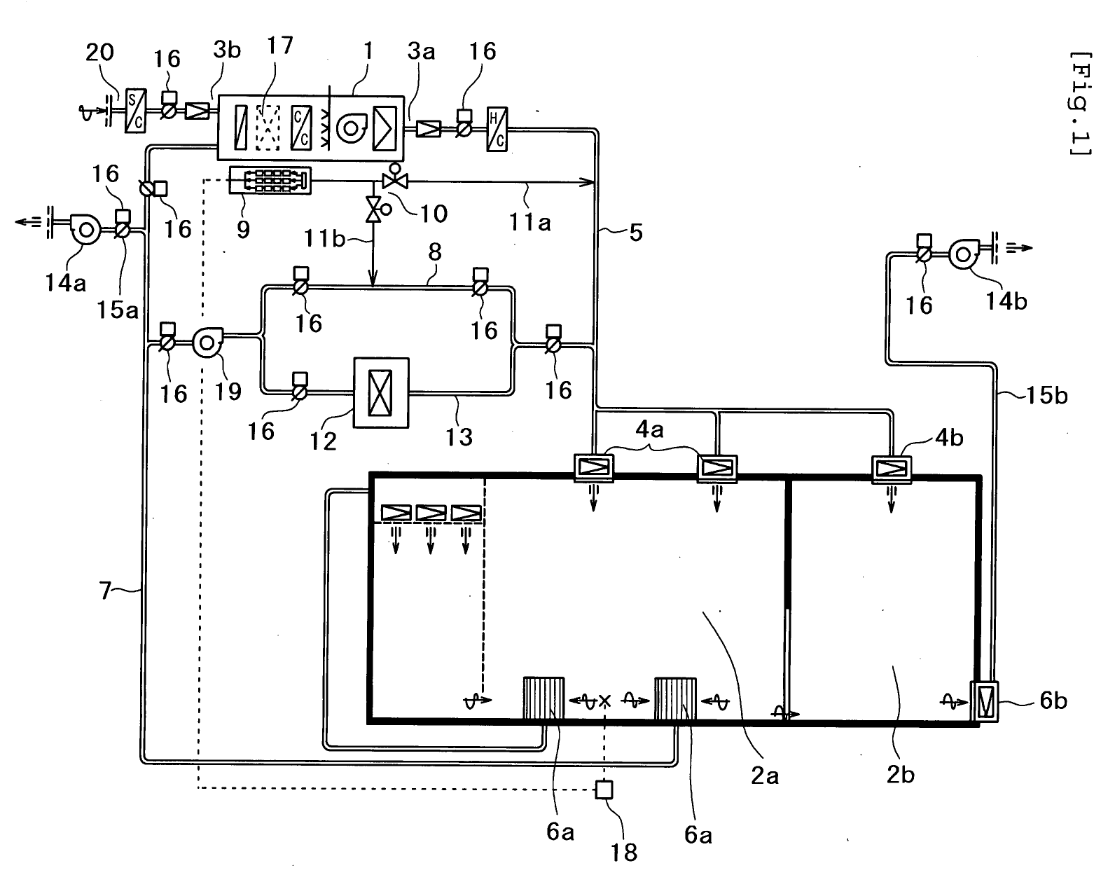

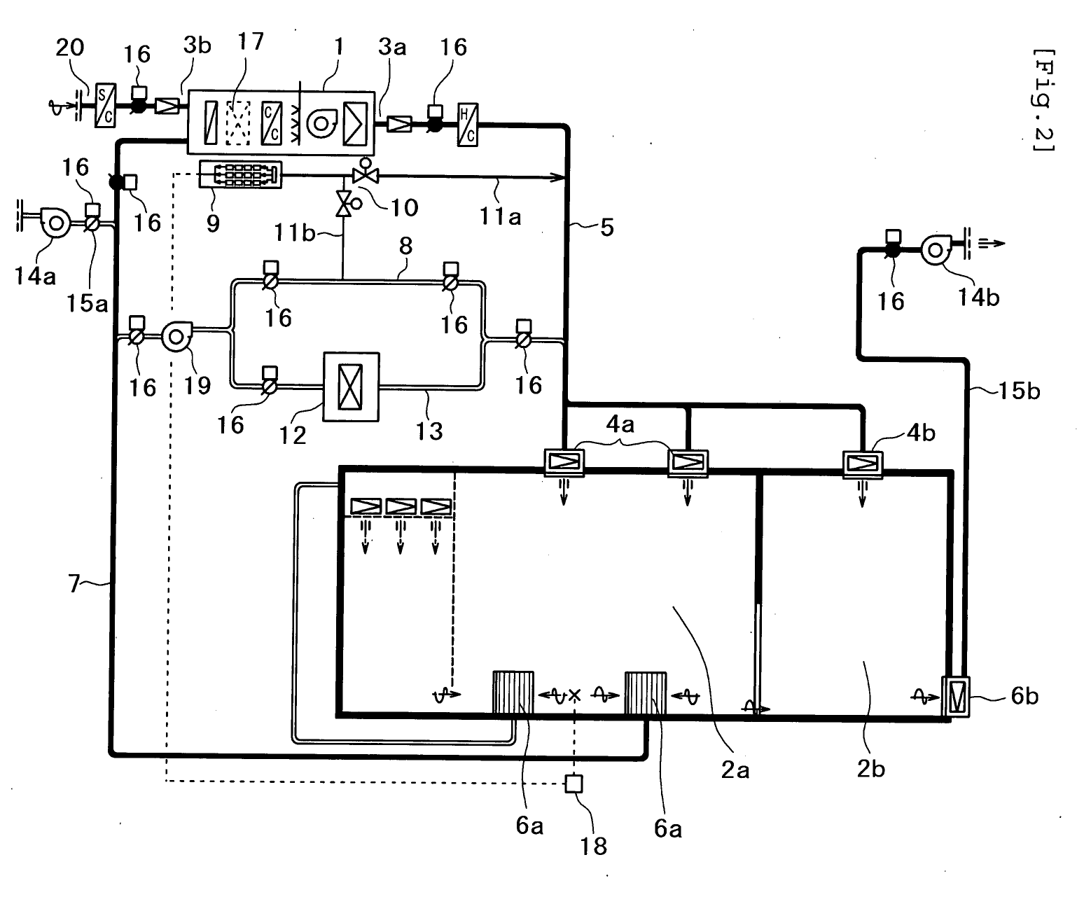

FIG. 1 is a system illustration showing an example of the system of this invention conceptually. Symbol 1 denotes an air conditioner, and 2 (2a, 2b, . . . ) denote rooms required to be sterilized. A conditioned air supply path 5 extends from the discharge side 3a of the air conditioner 1 to the air supply openings 4 (4a, 4b, . . . ) of the rooms 2 required to be sterilized, and an indoor air suction path 7 extends from the air suction openings 6 (6a, . . . ) of the rooms required to be sterilized to the suction side 3b of the air conditioner 1. The conditioned air supply path 5 and the indoor air suction path 7 constitute an air conditioning path. A bypath 8 is established to bypass the air conditioner 1. An ozone supply path extends from the ozone generation unit 9 to the switching mechanism 10 and further branches into ozone supply paths 11a and 11b connected with the conditioned air supply path 5 and the bypath 8 respectively. An ozone decomposition path 13 with an ozone decomposition unit 12 is established in parallel to the bypath 8. Further, an air discharge path 15a with an air discharge fan 14a is connected with the indoor air suction path 7, and an air discharge path 15b with an air discharge fan 14b is connected with the room 2b required to be sterilized. Moreover, a switching mechanism is provided for switching between the air conditioning path consisting of the conditioned air supply path 5 and the indoor air suction path 7, and the bypath 8, the ozone decomposition path 13 or the air discharge path 15a or 15b. The switching mechanism is actuated by opening and closing the many motor dampers 16 shown in the drawing. In the drawings after FIG. 2 inclusive, each damper denoted by a closed circle means that the damper is opened, and each damper denoted by a non-closed circle means that the damper is closed.

In this constitution, in FIG. 2, while the air conditioning path through the air conditioner 1 is selected by the switching mechanism, to perform air conditioning operation, ozone gas is supplied into the conditioned air supply path 5 from the ozone generation unit 9 through the ozone supply path 11a selected by the switching mechanism 10.

In this operation state, the ozone gas flowing into the conditioned air flowing in the conditioned air supply path 5 flows from the conditioned air supply path 5 into the rooms 2a and 2b from the air supply openings 4, for sterilizing the rooms 2a and 2b internally by fumigation.

Then, the ozone-containing conditioned air in the room 2a flows through the air suction openings 6a into the indoor air suction path 7 and flows from the indoor air suction path 7 to the suction side 3b of the air conditioner 1. After it flows into the air conditioner 1, it flows again from the discharge side 3a into the conditioned air supply path 5, being supplied again into the rooms 2a and 2b.

In this case, in the air conditioner 1, as required, the ozone decomposition catalyst 17 can be installed at a position upstream of the heat exchange coils. In this constitution, since the ozone in the conditioned air flowing into the air conditioner 1 from the suction side 3b is decomposed by the ozone decomposition catalyst 17 installed on the upstream side of the heat exchange coils, the corrosion of the heat exchange coils by ozone can be prevented. The ozone decomposition catalyst 17 is effective, in the case where the heat exchange coils are respectively made of a material likely to be corroded by ozone. The ozone decomposition catalyst 17 can be installed in the air conditioner as shown in the drawing, but can also be installed outside the air conditioner, namely, at a position upstream of the air conditioner, if it is installed at a position upstream of the heat exchange coils.

In this case, the conditioned air flowing from the discharge side 3a of the air conditioner 1 again into the conditioned air supply path 5 does not contain ozone. However, since ozone gas is supplied through the ozone supply path 11a into the conditioned air supply path 5, ozone is continuously supplied together with conditioned air into the rooms 2a and 2b, to perform sterilization by fumigation.

In the above, the amount of ozone supplied through the ozone supply path 11a into the conditioned air supply path 5 can be adequately adjusted based on the measured value of an ozone concentration meter 18 for measuring the ozone concentration at an adequate place in the room 2a.

Meanwhile, as shown in FIG. 2, from any room 2b of plural rooms 2a and 2b, the ozone-containing conditioned air can also be partially discharged through the air discharge path 15b by the action of the air discharge fan 14b.

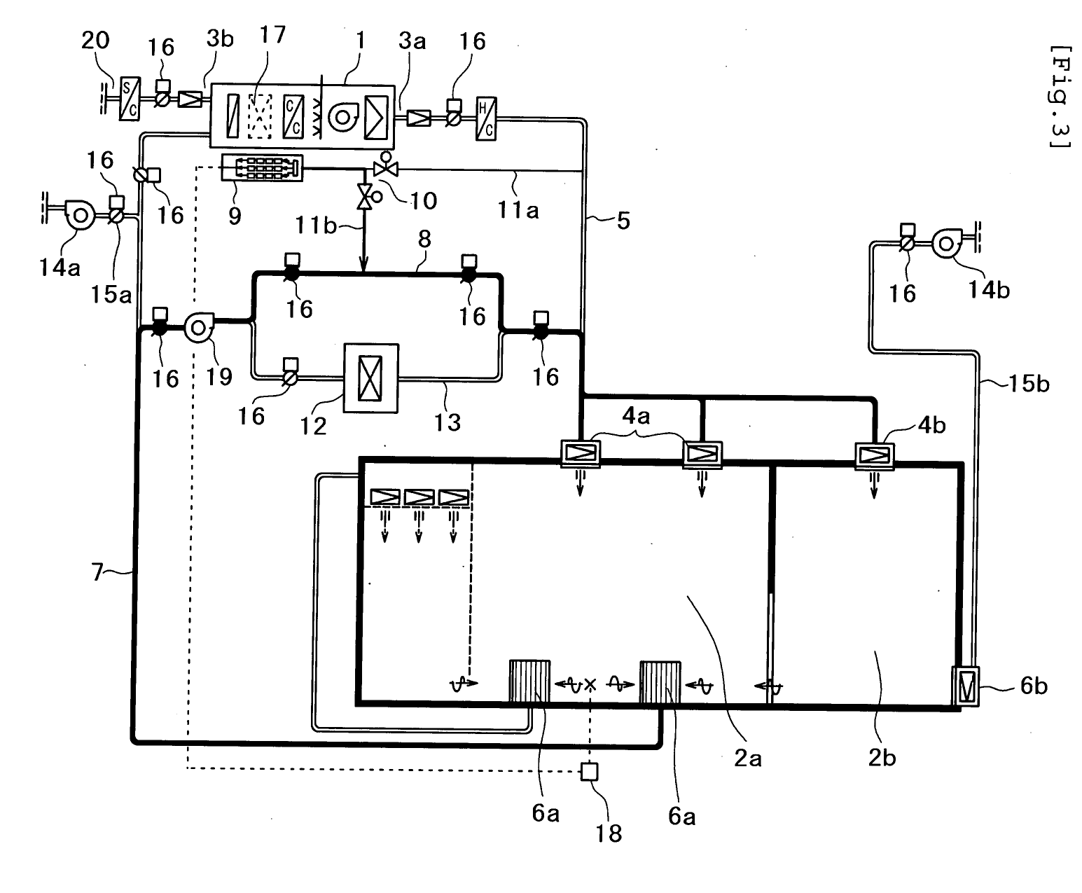

In FIG. 3, the bypath 8 for bypassing the air conditioner 1 is selected instead of the air conditioning path through the air conditioner 1, so that ozone gas can be supplied from the ozone generation unit 9 to the bypath 8 through the ozone supply path 11b selected by the switching mechanism 10.

In this state, by the fan 19, ozone is supplied into the air flowing in the bypath 8 from the ozone supply path 11b, and the ozone-containing air flows through the conditioned air supply path 5 into the rooms 2a and 2b from the air supply openings 4, for sterilizing the rooms 2a and 2b internally by fumigation.

Then, the air in the rooms 2a and 2b flow from the air suction openings 6a into the indoor air suction path 7 and flows from the indoor air suction path 7 to a position upstream of the bypath 8. Again in the bypath 8, ozone is supplied, and the ozone-containing air is supplied to the rooms 2a and 2b.

In the case of FIG. 3, while air conditioning is kept suspended, the rooms 2a and 2b and the piping system can be sterilized, and in this operation, since the ozone-containing air does not pass through the air conditioner 1, the ozone concentration in the rooms 2a and 2b can also be set at a higher level.

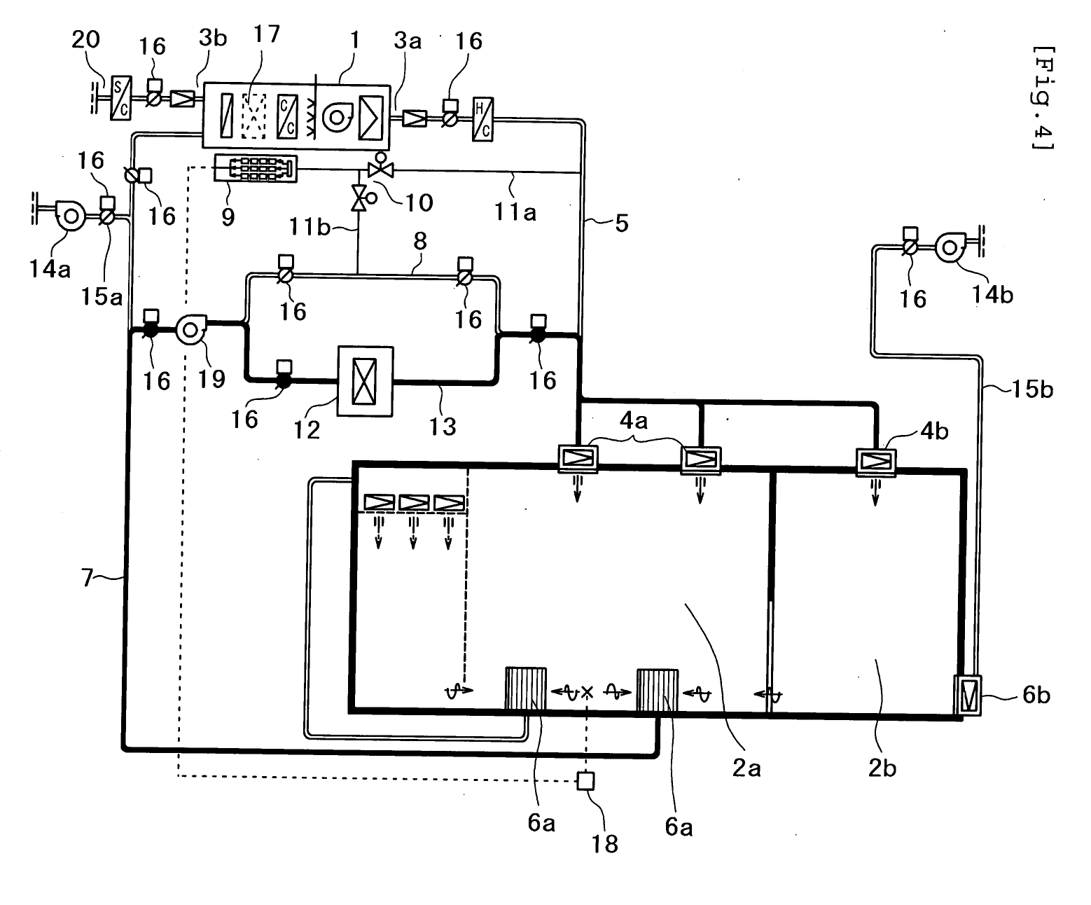

After the operation of FIG. 3 has been continued for an adequate period of time, the supply of ozone from the ozone supply path 11b is stopped in FIG. 4, and the switching mechanism is used to switch from the bypath 8 to the ozone decomposition path 13.

In this state, the ozone-containing air in the rooms 2a and 2b flows through the ozone decomposition path 13 and is decomposed by the ozone decomposition unit 12. Therefore, the ozone concentration in the rooms 2a and 2b gradually declines.

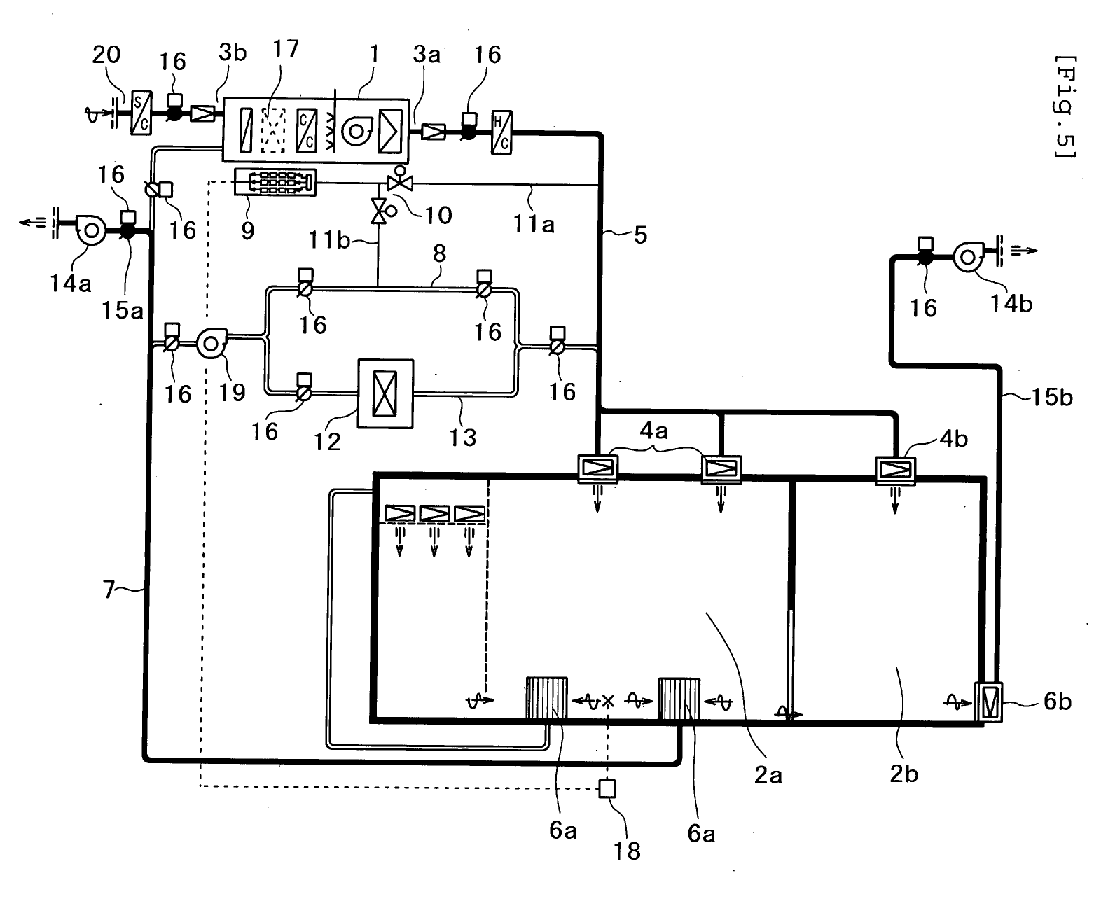

If the ozone concentration in the rooms 2a and 2b has declined to lower than a certain level in this way, then the air in the air conditioning system is discharged together with the air in the rooms 2a and 2b. For this discharge of air, as shown in FIG. 5, the conditioned air supply path 5 and the indoor air suction path 7 of the air conditioner 1 are partially used and the air exhaust paths 15a and 15b are also used though the bypath 8 and the ozone decomposition path 13 are not used. Further, an outdoor air introduction path 20 connected with the suction side 3b of the air conditioner 1 is used, but air is not returned through the indoor air suction path 7 to the suction side 3b of the air conditioner 1.

In the state of FIG. 5, the air in the rooms 2a and 2b is replaced by the outdoor air introduced through the outdoor air introduction path 20, and ozone can be perfectly discharged from the rooms 2a and 2b.

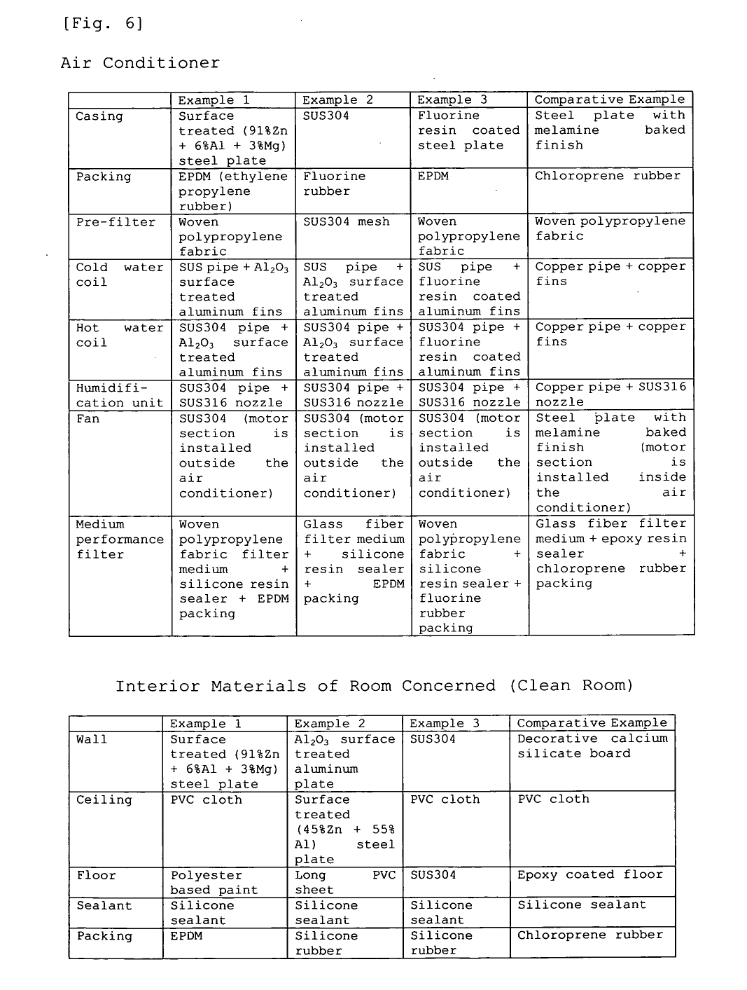

In this invention, in the air conditioning system operated as described above, the component materials destined to contact ozone gas, of the system, are materials with an ozone exposure resistance ratio of 0.75 or more in exposure to an ozone concentration of 200 ppm for 500 hours. Among the component materials, the resin materials used have a resin saturation degree of 70% or more. Among the component materials, inorganic materials are those, in the surfaces of which one or more of Al, Cr, Zn, Ni and Si account for 3% or more of the material concerned. Meanwhile, the component materials are shown in FIGS. 6, 7 and 8, and in addition, there are parts very small in contact area such as screws and sensors and easily exchangeable parts such as lighting fixtures and filters.

If adequate materials are selected as the component materials destined to contact ozone gas, of the system, the deterioration of materials can be prevented, and contaminants such as dust generated by the deterioration of materials can be reduced.

FIG. 6 shows examples of the interior materials of the air conditioner 1 and the rooms 2a and 2b together with comparative examples.

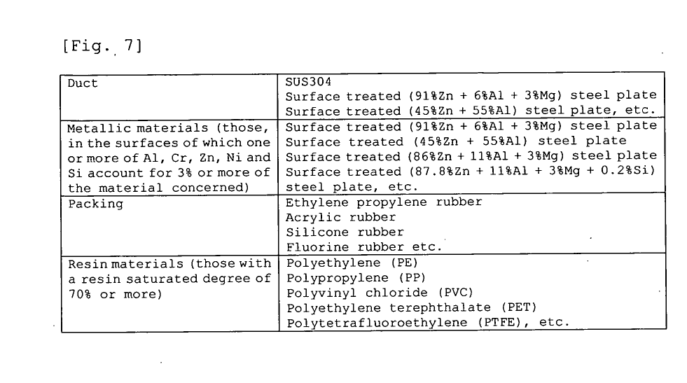

Further, FIG. 7 shows examples the respective materials

### INDUSTRIAL APPLICABILITY

Since this invention is as described above, a system capable of fumigating the rooms required to be sterilized using ozone free from residual tendency unlike conventional formaldehyde, can be constructed as an air conditioning system.

In the air conditioning system of this invention, while the air conditioning path through the air conditioner is selected by the switching mechanism, to perform air conditioning operation, ozone gas can be supplied from the ozone generation unit to the conditioned air supply path through the switching mechanism, allowing the rooms concerned to be sterilized by fumigation with ozone, and also allowing the air conditioning system including the air conditioner and the piping system as a whole to be sterilized.

In this case, if the ozone decomposition catalyst is installed upstream of the heat exchange coils as required, the corrosion of the heat exchange coils by ozone can be prevented.

Moreover in this invention, if the bypath for bypassing the air conditioner is selected instead of the air conditioning path through the air conditioner, ozone gas can be supplied from the ozone generation unit through the switching mechanism to the bypath, for allowing the rooms concerned and the piping system to be sterilized while air conditioning is kept suspended.

Further in this invention, if adequate materials are selected as the component materials destined to contact ozone gas, of the system, the deterioration of materials can be prevented, and contaminants such as dust generated by the deterioration of materials can be reduced.

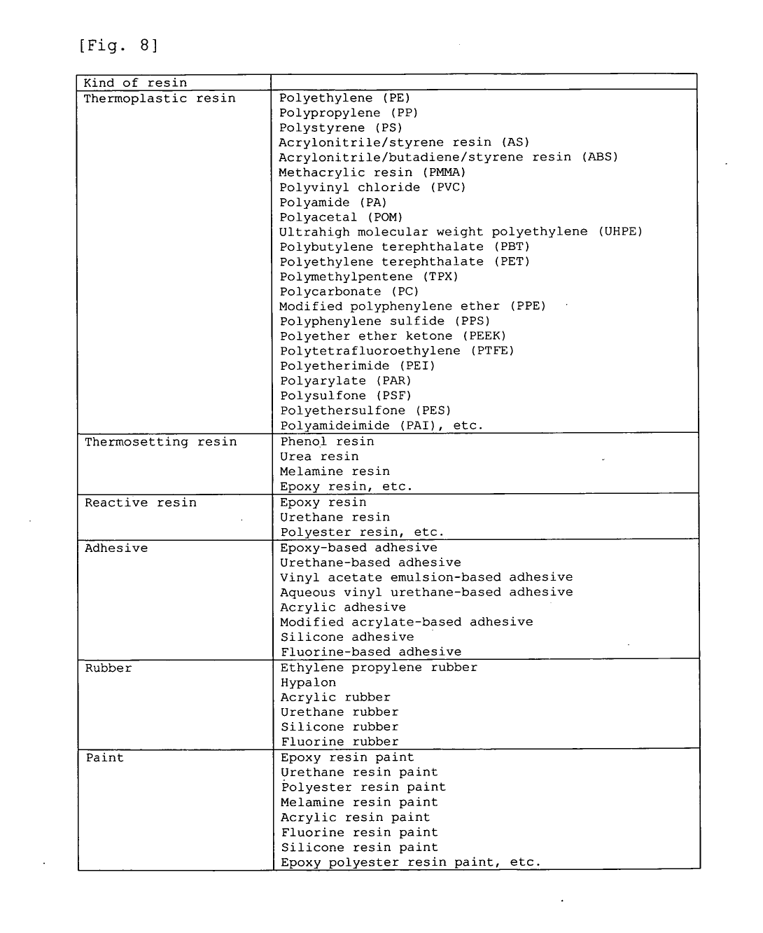

### BRIEF DESCRIPTION OF THE DRAWINGS

[FIG. 1] is a system illustration for conceptually showing the constitution of the air conditioning system for the rooms required to be sterilized, of this invention.

[FIG. 2] is a system illustration for conceptually showing the constitution of the air conditioning system for the rooms required to be sterilized, of this invention, in one mode of operation.

[FIG. 3] is a system illustration for conceptually showing the constitution of the air conditioning system for the rooms required to be sterilized, of this invention, in another mode of operation.

[FIG. 4] is a system illustration for conceptually showing the constitution of the air conditioning system for the rooms required to be sterilized, of this invention, in a further other mode of operation.

[FIG. 5] is a system illustration for conceptually showing the constitution of the air conditioning system for the rooms required to be sterilized, of this invention, in a still further other mode of operation.

[FIG. 6] shows examples of the components of the air conditioning system for the rooms required to be sterilized, of this invention.

[FIG. 7] shows examples of the components of the air conditioning system for the rooms required to be sterilized, of this invention.

[FIG. 8] shows examples of the components of the air conditioning system for the rooms required to be sterilized, of this invention.

MEANINGS OF SYMBOLS

| | |
|---|---|
| 1 | air conditioner |
| 2 (2a, 2b) | rooms required to be sterilized |
| 3a | discharge side |
| 3b | suction side |
| 4 (4a, 4b) | air discharge opening |
| 5 | conditioned air supply path |
| 6 (6a, . . .) | air suction opening |
| 7 | indoor air suction path |
| 8 | bypath |
| 9 | ozone generation unit |
| 10 | switching mechanism |
| 11a, 11b | ozone supply paths |
| 12 | ozone decomposition unit |
| 13 | ozone decomposition path |
| 14a, 14b | air discharge fan |
| 15a, 15b | air discharge path |
| 16 | motor damper |
| 17 | ozone decomposition catalyst |
| 18 | ozone concentration meter |
| 19 | fan |
| 20 | outdoor air introduction path |

1. An air conditioning system for the rooms required to be sterilized, comprising:
an air conditioning path consisting of a conditioned air supply path extending from the discharge side of an air conditioner to the air supply openings of the rooms required to be sterilized;
an indoor air suction path extending from the air suction openings of the rooms required to be sterilized to the suction side of the air conditioner;
a bypath for bypassing the air conditioner;
an ozone supply path extending from an ozone generation unit to a switching mechanism and further branching into the ozone supply paths connected with said conditioned air supply path and said bypath;
an ozone decomposition path with an ozone decomposition unit, in parallel with the bypath;
an air discharge path with an air discharge fan, connected with said indoor air suction path or with the rooms required to be sterilized; and
a switching mechanism for switching between said air conditioning path, said bypath, said ozone decomposition path and said air discharge path.

2. An air conditioning system for the rooms required to be sterilized, according to claim 1, wherein an ozone decomposition catalyst is installed at a position upstream of the heat exchange coils of the air conditioner.

3. An air conditioning system for the rooms required to be sterilized, according to claim 1, wherein materials with an ozone exposure resistance ratio of 0.75 or more in exposure to an ozone concentration of 200 ppm for 500 hours are used as the component materials destined to contact ozone gas, of the system.

4. An air conditioning system for the rooms required to be sterilized, according to claim 1, wherein resin materials with a resin saturation degree of 70% or more are used as the resin materials destined to contact ozone gas, of the system.

5. An air conditioning system for the rooms required to be sterilized, according to claim 1, wherein inorganic materials, in the surfaces of which one or more of Al, Cr, Zn, Ni and Si account for 3% or more of the material concerned, are used as the inorganic materials destined to contact ozone gas, of the system.

6. An air conditioning system for the rooms required to be sterilized, according to claim 2, wherein materials with an ozone exposure resistance ratio of 0.75 or more in exposure to an ozone concentration of 200 ppm for 500 hours are used as the component materials destined to contact ozone gas, of the system.

7. An air conditioning system for the rooms required to be sterilized, according to claim 2, wherein resin materials with a resin saturation degree of 70% or more are used as the resin materials destined to contact ozone gas, of the system.

8. An air conditioning system for the rooms required to be sterilized, according to claim 2, wherein inorganic materials, in the surfaces of which one or more of Al, Cr, Zn, Ni and Si account for 3% or more of the material concerned, are used as the inorganic materials destined to contact ozone gas, of the system.
